(19) United States
(12) Patent Application Publication
(10) Pub. No.: US 2020/0254310 A1
(43) Pub. Date: Aug. 13, 2020

(54) ADAPTIVE VIRTUAL REHABILITATION

(71) Applicant: Triad Labs, LLC, New Concord, OH (US)

(72) Inventors: Larry Triplett, New Concord, OH (US); Greg Adams, New Concord, OH (US)

(21) Appl. No.: 16/789,743

(22) Filed: Feb. 13, 2020

Related U.S. Application Data
(60) Provisional application No. 62/804,990, filed on Feb. 13, 2019.

(51) Int. Cl. A63B 24/00; A61B 5/00; A61B 5/11; G16H 20/30; G06N 20/00; G09B 9/00

(52) U.S. Cl. CPC A63B 24/0075; A61B 5/4824; A61B 5/1124; A61B 5/7267; A63B 2071/0652; A61B 5/486; G16H 20/30; G06N 20/00; G09B 9/00; A61B 5/7445

(57) ABSTRACT

A system and method (referred to as the system) render treatment plans through virtual rehabilitation by executing, a physical assessment that captures a full volume of motion of an uninjured appendage or a comparable standard. The process generates a recovery path, care plan, a challenge zone for an injured appendage based on the volume of motion of the uninjured appendage and initiates a virtual therapeutic session that renders simulated virtual environments in that causes the user to exercise the injured appendage within the challenge zone. The process analyzes the user's virtual therapeutic session performance by comparing a user's volume of motion capacity to the user's full volume of motion.

### PRIORITY CLAIM

This application claims priority to U.S. Provisional Patent Application No. 62/804,990, titled System and Method for Virtual Reality Enhanced Adaptive Rehabilitation Feb. 13, 2019, which is herein incorporated by reference.

### BACKGROUND OF THE DISCLOSURE

### 1. Technical Field

This application relates to rehabilitation, and particularly to virtual rehabilitation.

### 2. Related Art

As science and technology advances, so does medicine. Technology allows users to explore simulated environments allowing users explore their anatomy, for example, and train by attempting simulated surgical procedures. The environments are often limited and not well suited to distributed processing.

Many computer generated environments are inaccessible to users as they require specialized interfaces that do not accommodate their needs or their medical conditions. Some equipment reduces the users' natural reactions because it is not tailored to the users' special needs. Children diagnosed with hyperactivity, for example, may not adapt to adult-sized equipment.

Another concern is safety. When equipment is used to rehabilitate an injury, for example, any movement presents a risk of reinjury due to its setup (some are tethered to other equipment or weighted), a user moving beyond prescribed therapy levels and/or the user engaging beyond prescribed therapy sessions. Similarly, not moving to prescribed levels or engaging for extended time periods can also cause injury.

### BRIEF DESCRIPTION OF THE DRAWINGS

The patent or application file contains at least one drawing executed in color. Copies of this patent or patent application publication with color drawing(s) will be provided by the Office upon request and payment of the necessary fee. The elements in the figures are not necessarily to scale, emphasis instead being placed upon illustrating the principles of the disclosure. Moreover, in the figures, like referenced numerals designate corresponding parts throughout the different views.

FIG. 1 is a virtual rehabilitation process that processes feedback in a virtual environment.

FIG. 2 shows representations of the volume of motion full capacity, the volume of motion current capacity, and a game play challenge zone.

FIG. 3 shows representations of a user's performance during a virtual therapeutic session with respect to their volume of motion full capacity, their volume of motion current capacity, and the game play challenge zone.

FIG. 4 is a graphical user interface that provides a means for selecting one of several options in a care plan.

FIG. 5 is a graphical user interface of the user's preselection settings.

FIGS. 6-10 show the various reports that may be generated by the systems. For each of the five volumes in FIG. 6, the goals and actual volumes may be shown in separate windows.

FIG. 11 is a virtual rehabilitation system.

### DETAILED DESCRIPTION

The disclosed systems and processes (referred to as systems) render new and unique treatment plans through virtual rehabilitation. The systems provide rehabilitation programs by enabling users to move and react to computer-simulated virtual environments. The systems allow users to sense, move, and/or influence virtual objects much like they do to physical objects in a natural real-life environment. The natural flow and interactions of some therapeutic activities provide entertainment and real-time movements without revealing to the user that the user is undergoing therapy meant to restore their good health. The immersion into simulated environments allow users to forget about their surroundings, health, and other external situations. Some simulated environments create sight sensations that encourage physical movements. Others, transmit sight, sound, and sensations that simulate the real world making the simulations different from traditional computer simulations. Some systems record movements, and some alternate systems record and track sound, facial expressions, movements and/or positions. The systems monitor and assess progress as users move through rehabilitative programs while ensuring user safety. Alternate systems include safety monitoring programs that guard against reinjury by processing feedback and adjusting the program in real-time when users exercise beyond their prescribed activities, beyond their prescribed durations, or express pain. A real-time operation comprises an operation matching or occurring faster than a human's perception of time (e.g., processing information or data at the same rate or at a faster rate than it is received) or a virtual process that occurs (or is perceived to occur) like a process in a real life environment.

In some systems, the virtual worlds are generated by mathematical models and programming that creates the illusion of motion, the appearance of objects, and their motion and/or removal. By manipulating the transitions between scenes, and recalculating positions between them, the systems provide the illusion of continuous motion of virtual objects. Some processes calculate the viewer's perspective of the virtual object, calculate lighting, add shadings, reflections, shadows, and textures to simulate the manipulation of what appears to be real life objects. By specifying motions, appearances, and transparency (virtual object removal) scenes are rendered and action specified through displays such as through a head-mounted display.

To see in the virtual environment, the users wear a head-mounted display with screens directed to each of the user's eye. The head-mounted display includes a position tracker to monitor the location and movement of the user's head, and appendages and in alternate systems, include vision sensor and microphones to track the user's eyes, facial expressions, and sound. With respect to the tracking of the user's head and eyes, the systems process position data to recalculate the images rendered in the virtual environment to match the direction in which the user is looking and displaying those images on the head-mounted display. Using a plurality of sensors, such as optical sensors, capacitive sensors, and/or force sensors, for example, the systems detect a volume of a motion cloud in which an appendage moves. This is the amount of space or cloud of space in which the appendage can move. When improving the motor training of an appendage extension or extension motion, the cloud of space comprises the amount of spatial volume in which an appendage can reach out, extend, and move. It may encompass the spatial volume that allows a user to point, reach, or extend toward an object or simulate the spatial volume required to point to, throw, or throw at an object. By employing a very high sampling rate (e.g., some systems track motion coordinates about every 100 milliseconds), the systems capture subtle gestures and track their movements in these spatial volumes that provide a very high granular measurement of the user's range of motion. This is valuable in therapy sessions where incremental movements can make the difference between a successful rehabilitation therapy and an injury. In some systems, appendage tracking, such as arm and hand tracking, for example, may occur through one or more optional handheld devices in wireless communication with the head-mounted display. The optional handheld devices may be adjusted to different hand sizes, and in some systems, be made with anti-microbial surfaces to maintain sterile environments.

In some systems, pain is tracked by monitoring sound (e.g., within or outside of the aural range), facial expressions, and head movements via a pain tracking engine. Because pain often coincides with a facial expressions and head movements, active model are used in alternate systems that compare features extractions from motion analytics in which the user is in pain with pain-free features extracted from benchmark data and user head motions and rates of those movement against head motions and rates of those motions associated with pain. In other alternate systems, pain may be recognized through voice recognitions that act on verbal commands, verbal expressions, or other sounds to detect the user's sound inflections that are often associated with pain through the pain tracking engine. In these alternate systems, when pain is recognized (or a pain level is reached) the alternate systems initiate assessments and other some systems initiate remedial actions such as terminating the interactive virtual therapeutic session to prevent reinjury and/or initiate real time adjustments to achieve the most impactful recovery path and care plan.

In some fully immersive systems, the user's may hear sounds via speakers, such as the popping of a balloon in an interactive virtual therapeutic session. And, in some alternate systems, the user may sense touch that is simulated by physical feedback via a haptic interface in the handheld devices. The haptic interfaces relay feedback (that in other systems include sound and/or visual cues), such as when a user is making an unprescribed movement and/or engaging beyond a prescribed therapy session to provide an alert or notification. The alert or notification that may also be relayed to the therapist via an alert engine and a messaging systems. Some haptic interface relays physical sensations that coincide with the limits and boundaries in the virtual environment.

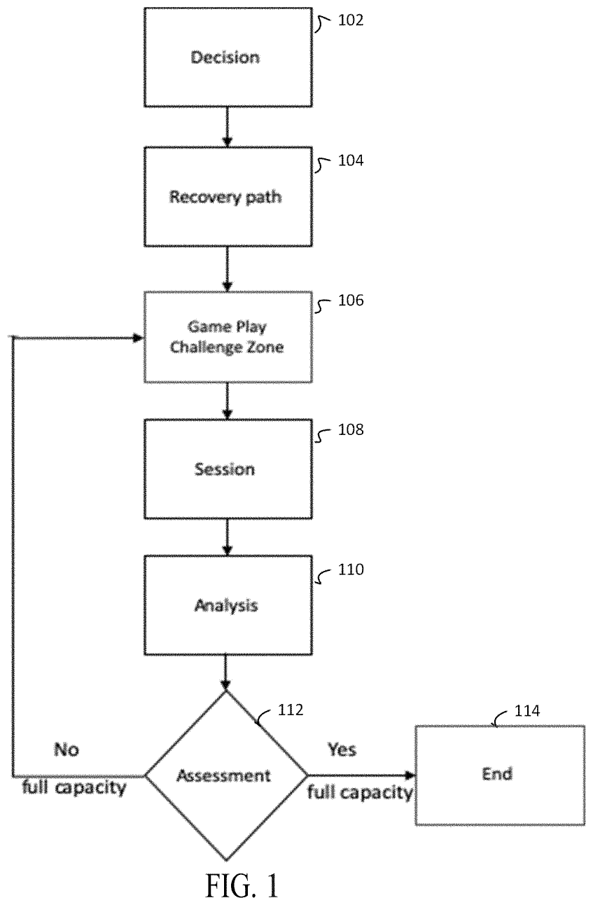

FIG. 1 is a virtual rehabilitation process that processes feedback in a virtual environment. At 102 an initial assessment and diagnosis is made by capturing the initial volume of motion (VOM) in memory. While the virtual simulations and virtual environments may differ depending on the selected therapeutic program, benchmarks can be set by user motions, clinical observations, baselines of similar measures, or historical data. For example, if a user injured an arm, a benchmark may be established by having the user engage a virtual environment with their uninjured arm allowing the system to track the user's arm motions in three dimensions (e.g., via x, y, and z coordinates or three dimensional vectors). The system thereafter maps the user's arm motion to a three dimensional space quantified by a volume measurement. A volume is the amount or region of space a three-dimensional object (here, an arm) moves through expressed in cubic units. Alternatively, benchmarks may be established by observation, historical data, baselines of similar measures, or a desired target volume of a motion level the episode of care desires to achieve.

With the initial assessment completed, a dynamic active care plan engine (e.g., a machine learning trained engine) generates a recovery path and care plan at 104 that may be based on the therapist or therapeutic team's recommendations. The care prescribed and speed of the recovery path may be based on the user's diagnosis (e.g., a rotator cuff injury may require a less aggressive therapy than an overextended muscle), current health, desired rehabilitation period, and other factors.

In preparation for the virtual therapeutic session, the systems process the care plan and assessments made from any prior therapeutic sessions. For physical rehabilitation, the systems measure various distances. For a shoulder rehabilitation, for example, the systems measure the distances of the user's arms between their joints. The distances are translated into three dimensional virtual environment vectors that establishes a starting rehabilitative cloud centered around the simulated user. The systems also align the virtual environment vectors with prescribed range of motion established by the care plan.

Based on the care plan, a game play challenge zone is created at 106. A game play challenge zone comprises an area, such as a spatial volume in the virtual environment in which a user attempts to move part of his/her virtual self. Movement in the virtual environment is linked to the user's relative movement of the handheld or other device, but not to the precise physical location of the device. For example, if a user picks up the device without extending his/her arm during an arm reach rehabilitation therapy and then sets it down in another location, the representation or motion of the user in the virtual environment does not change because no arm extension was detected. When the user executes an extension, the movement is reflected by a motion in the virtual environment to reflect the user's extension relative to their starting position. A virtual therapeutic session is the time during which a program is running. In some systems, it is the time during which the user interacts within the interactive environment which may be described as the time during which the program accepts rehabilitative input from the user and processes that information.

At 108 the virtual therapeutic session begins. In some systems, the virtual therapeutic session occur for a predetermined amount of time that may occur on scheduled number of days of the week. For example, a virtual therapeutic session may last thirty minutes a day three to five days a week. During virtual therapeutic sessions, the user executes certain motions in response to requested activities prompted by the virtual world. A motor training of an appendage extension, for example, may be prompted by virtual reality exercises that encourage an extension and in some applications a desired trajectory to complete a task or movement. The virtual reality exercises recruit the user to move his/her injured appendage into the challenge zone throughout the virtual therapeutic session. The system records the location of patient's appendage at a predetermined rate to determine if the user is able to reach the prescribed targets. The movements are measured and translated into vector movements during the virtual therapeutic session.

A game play challenge may present a challenge that requires a user to reach or extend to as many balloons (targets) as possible that surround their virtual presence after reaching or extending toward them first with their healthy appendage (establishing a benchmark). The system analyzes the user's performance during the therapeutic session by comparing the user's current volume of motion capability to their full volume of motion capability at 110. When a desired capability is reached and maintained for a predetermined number of virtual therapeutic sessions 112, the virtual therapeutic session and episode (of care) terminates at 114. An episode refers to the length of time in which the user undergoes therapeutic activities to rehabilitate an injury. It comprises the total number of virtual therapeutic session required to execute the care plan.

Unlike conventional therapeutic approaches that rely on ranges of motion, the disclosed system process volumes of motion for motor training such as the motor training of an appendage. In this application, the volume of motion is described by the perimeter of the volume that a user's appendage can move. The system monitors this cubic range by recording points and vectors in three-dimensional space that a user (a.k.a. a patient in some field of use applications) can reach as measured between the head-mounted display and the wireless devices.

As explained, the system analyzes the user's current volume of motion capability against their volume of motion full capacity. The volume of motion full capacity is the volume of motion that a user can move their unimpaired arm/appendage or is a final targeted range based on other factors. This is the full range of motion that the system is attempting to achieve during the episode of care. Based on a current volume of motion capability, the system measures the progress or lack thereof that the user makes and provides that assessment to the dynamic active care plan engine as feedback. Based on the feedback, the dynamic active care plan engine modifies the challenge zone and volume of motion cloud for the next virtual therapeutic session to ensure that the user progresses to the desired target level during the episode of care. The volume of motion cloud is the space (a.k.a. the cloud space) in which a movement can be made. When executing a training of an appendage motion, for example, the volume of motion cloud determines the area the user can manipulate their arm. Should the user reach beyond the boundary, a safety monitoring program intervenes to prevent reinjury in some alternate systems.

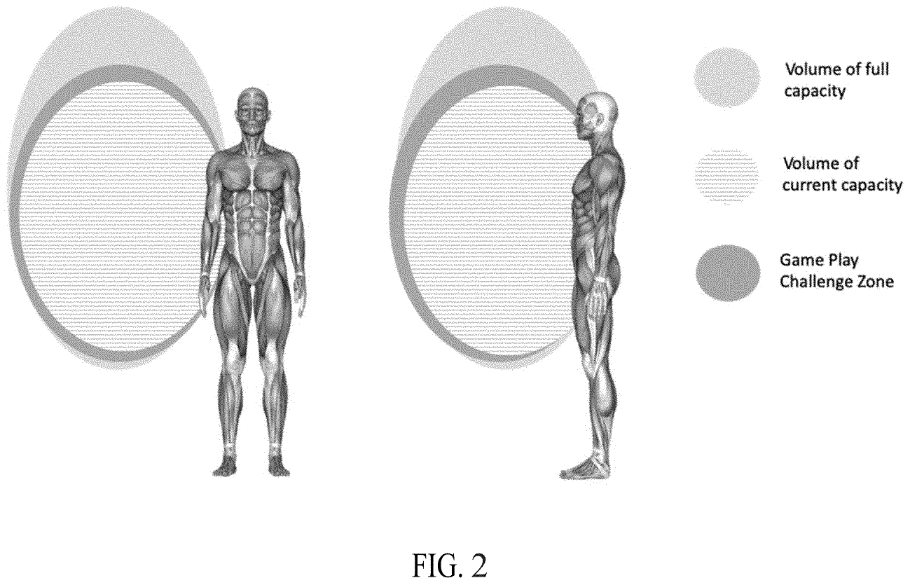

Each virtual therapeutic session establishes zones or boundaries and difficulty levels. The metrics are analogous to a perimeter for a real geographic area and a resistance training. In FIG. 2, the zones are referred to as game play challenge zones generated from high range intensity level data generated by a game difficulty level algorithm described below, a date, a time, a user identification, an episode identification, and the user's benchmark. In some use cases (such as training an appendage extension, for example), the benchmark represents the volume of space in which a user can move their uninjured appendage or some other comparable standard.

By an aggregation of movement data from all participants of the systems (impaired and fully functional in the appendage extension use case, for example,) retained in an on-line database, the game play zone algorithm matches participants with similar demographics and those with similar impairments as measured during each of the monitored virtual therapeutic session to identify similar prognoses. Based on those comparisons, the game play zone algorithm identifies assessments, and identifies users' virtual rehabilitation experiences, point targets, and movement patterns over time who exhibit the best progress in increasing and/or improving their volume of movement. In some exemplary unsupervised machine learning applications, the best practices render training data that is processed to train the dynamic active care plan engine. The training data may represent three dimensional coordinates (e.g., x, y, and z coordinates) of low and high limit clouds for participants and the percentage of points that should be applied in a game play zone (representing how aggressive the experience should be). The dynamic active care plan engine is trained on this data to minimize a loss function—such as a mean squared error (when mean is used) or mean absolute error (when median is used), for example,—by recursively splitting each of the many classes of the training data (e.g., three in this exemplary system) in a way that maximizes the best practices until an accuracy limit or threshold is met for each category. Thereafter, the trained dynamic active care plan engine establishes the game play zones as shown in FIG. 2.

A game difficulty level algorithm enhances the level of difficulty for the users while the user is engaging in the game play challenge zones. By processing the performance on their most current clinical range of motion assessments, their goal range of motion performances assessments, (from the recovery path of the episode of care), variances that exceed care plan recovery paths, and their historical virtual rehabilitation assessments, the systems generates game level difficulty levels. The levels are rendered as prescribed high range intensity levels (preferably in percentages where 100%=full range of motion). The game difficulty level algorithm further processes the current date and/or time, most recent pain level indicators, user identifications, and episode identifications.

In use, each of the virtual worlds may prompt users to target points and follow trajectories. When target points are rendered, the systems prompt users to reach to or extend to specific points in their physical space by prompting them to reach for extend toward renderings in their virtual worlds (e.g., a reach task). The user's performance is processed by the game play zone algorithm and game difficulty level algorithm to determine the game play challenge zones for future virtual therapeutic sessions. Target vector movements in the game play zone prompt the user to make specific movements along desired therapy trajectories from a starting point to an ending point. The user's progress is processed by the game play zone algorithm and game difficulty level algorithm to determine game play challenge zones for future virtual therapeutic sessions too. Because recommendations occur automatically in some systems, a virtual menu generator determines the correct menu choices available to the user based on the user's recovery path, current assessment, and prior performance from earlier virtual therapeutic sessions.

FIG. 2 shows representations of the volume of motion full capacity, the volume of motion current capacity, and the game play challenge zone. With respect to an arm extension, the volume of motion full capacity shown as a lighter shade of gray is the volume of space in which a user can move their unimpaired arm or another comparable arm. It is the volume of space the system attempts to attain during the episode of care. The volume of motion current capacity represented as a line space is the volume of space in which the user can currently move their impaired arm. The game play challenge zone shown as a darker shade of gray is the zone that the user attempts to attain during a virtual therapeutic session.

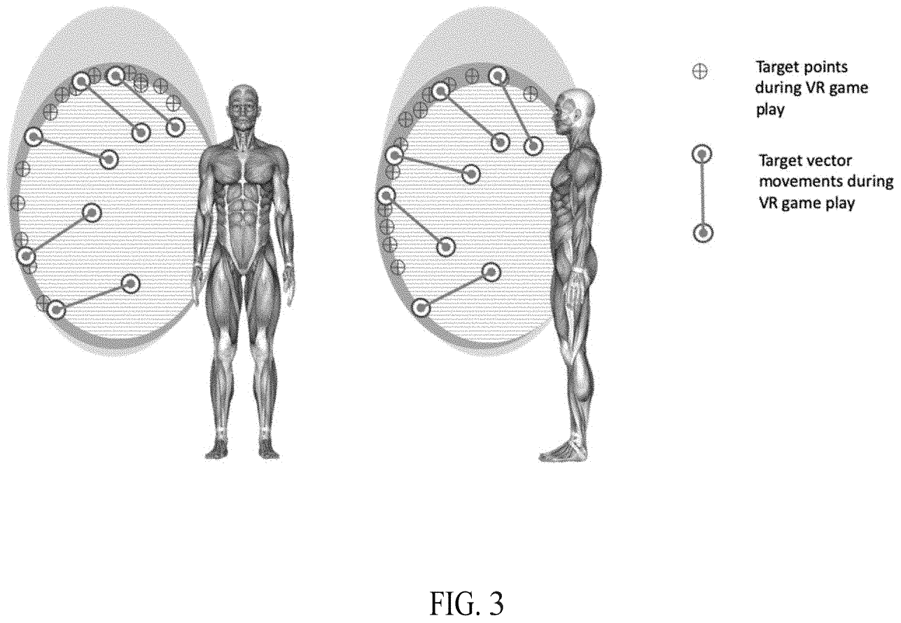

FIG. 3 shows representations of a user's performance during a virtual therapeutic session with respect to their volume of motion full capacity, their volume of motion current capacity, and the game play challenge zone. The operator symbol (the circle with an “X” drawn through it) represents the target extensions the system is encouraging the user to attain. While the target movement vectors represent the motion trajectory the user achieved during their virtual therapeutic session.

In practice, the volume of motion current capacity will improve or decline after each virtual therapeutic session. As a result, the improvement or decline is processed by the dynamic active care plan engine which automatically modifies the user's recovery path and care plan to reflect their current health. As a result, the game play challenge zone may also change for the next virtual therapeutic session. If little progress is recognized or perceived over a predetermined number of sessions, the alert engine may provide the user and/or therapist with a notification (e.g., an audible, haptic, or visual alarm) in some systems to indicate or report the lack of progress, which may further indicate that the user is not rehabilitating their injury to their full abilities for personal or financial reasons, for example, or the rehabilitation is not remediating the injury. The notification allows for adjustments that may improve the quality of care and outcomes, and reduce healthcare costs.

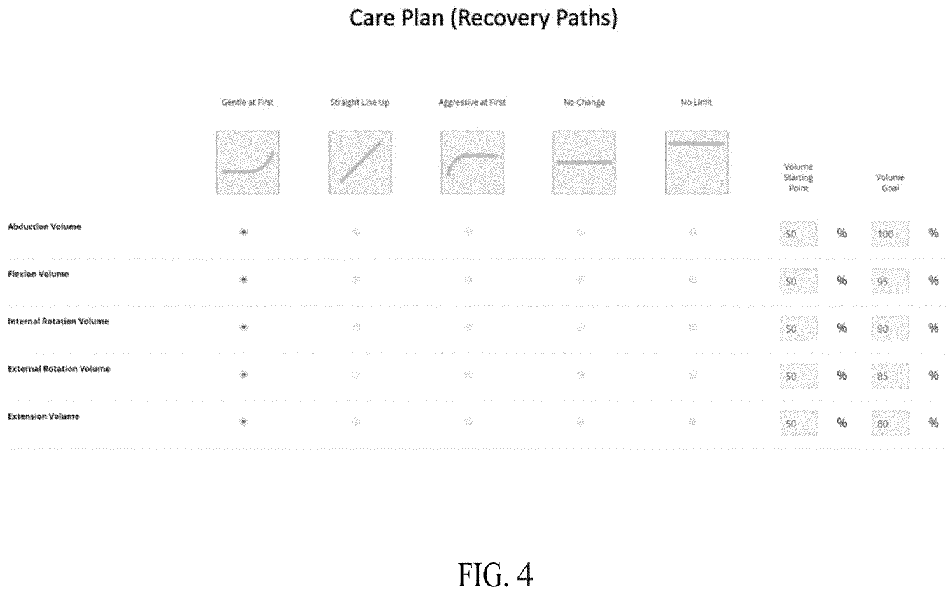

FIG. 4 is an optional graphical user interface that provides a means for selecting one of several options, within an option-selection area and dialog boxes adjacent the radio buttons that serve as a selector in some systems. A user or therapist may select five different volumes for the system to engage in by selecting a setting that deselects the remaining buttons in that row. The radio buttons appear as small circles that, when selected, has a smaller circle filled in. Each row is associated with a volume, volume starting point dialog box, and a volume goal dialog box. The spatial volume beside the user is the abduction volume. The spatial volume in front of the user is called the flexion volume. The user's turning is described by their internal rotation volumes, external rotation volumes, and spatial volumes behind the user are called extension volumes. The icons above the radio buttons represent the various intensity levels the user or therapists may elect for the various volumes of motion. The intensity levels reflect variable rates, linear rates, unchanging rates, and no limits. The dialog boxes adjacent the radio buttons establish the starting volumes and goals that may be used by the dynamic active care plan engine in each virtual therapeutic session, or in alternate systems episode of care.

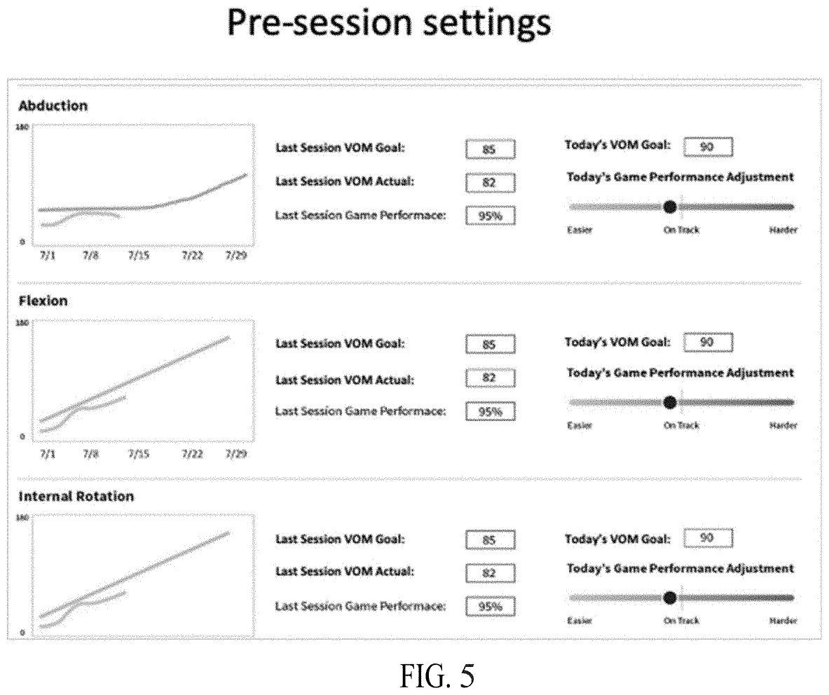

FIG. 5 is a graphical user interface of the user's preselection settings. For each of the five different volumes described above, the graphical user interfaces summarize their goals, actual ranges, and last virtual therapeutic session ranges of motion performance. In FIG. 5, the game play zone algorithm and the game difficulty level algorithm determine the area and difficulty level for the next virtual therapeutic sessions. In some systems, clinicians adjust the settings.

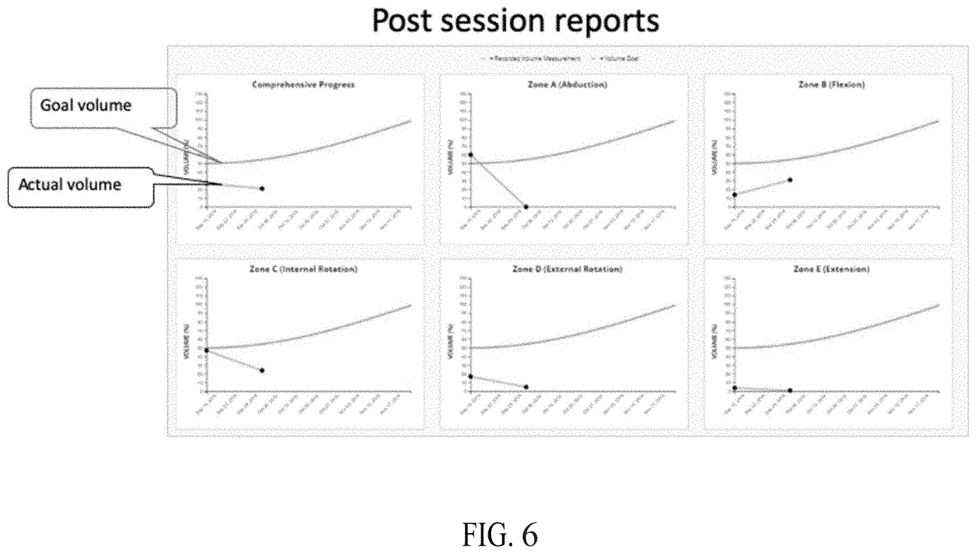

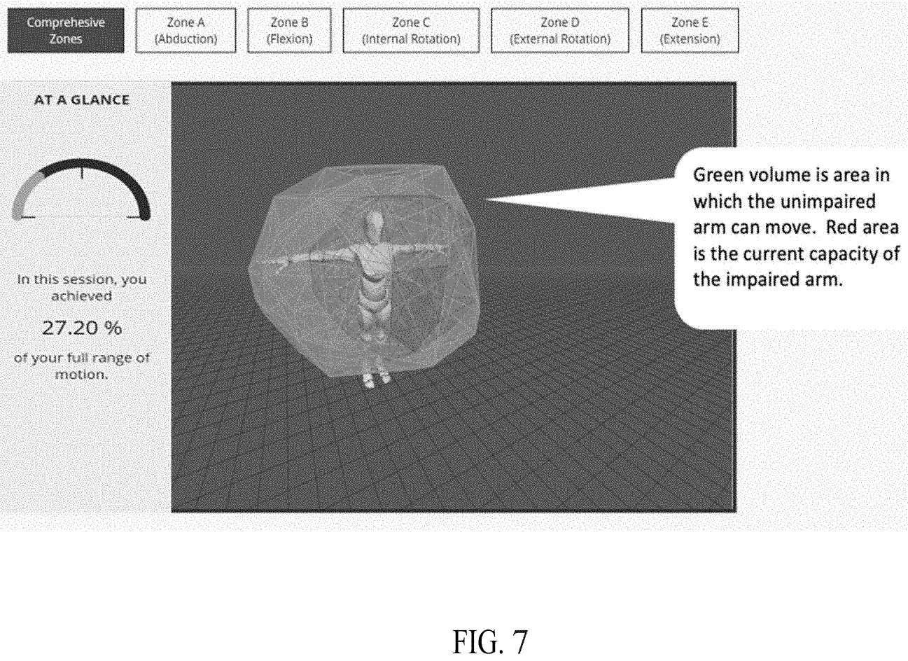

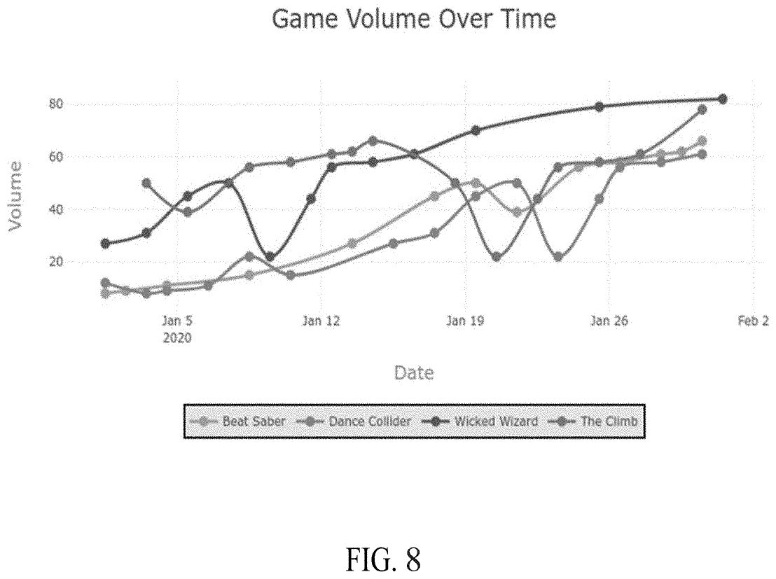

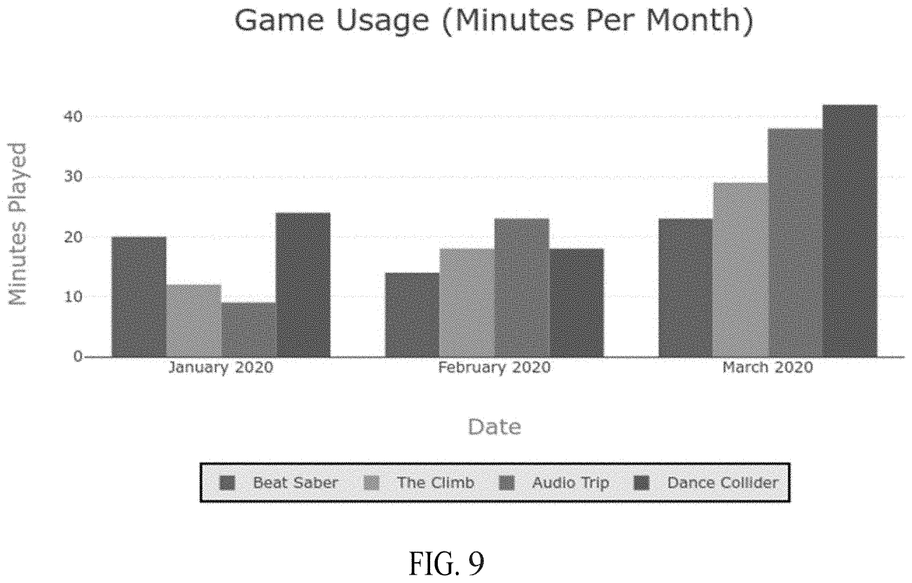

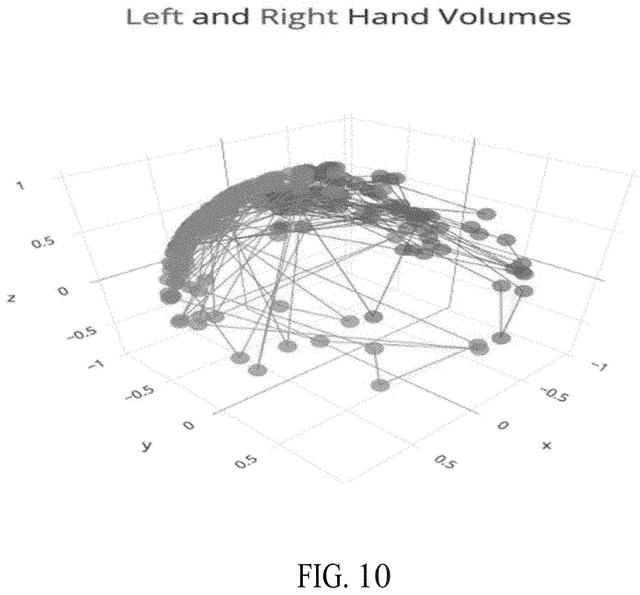

FIGS. 6-10 show the various reports that may be generated by the systems. For each of the five volumes in FIG. 6 the goals and actual volumes may be shown in separate windows. In FIG. 7 the green volume is area in which the unimpaired arm can move in an appendage extension exercise. The red area represents the current capacity of the impaired arm. FIGS. 8 and 9 show game volume over time with respect to select rehabilitative programs (e.g., Beat Saber, Dance Collider, Wicked Wizard, The Climb, and Auto Trip) and the minutes played per month. FIG. 10 shows comparisons to left and right hand volume. Other reports show volume performance contrasted with demographic averages and performances within similar dimensions.

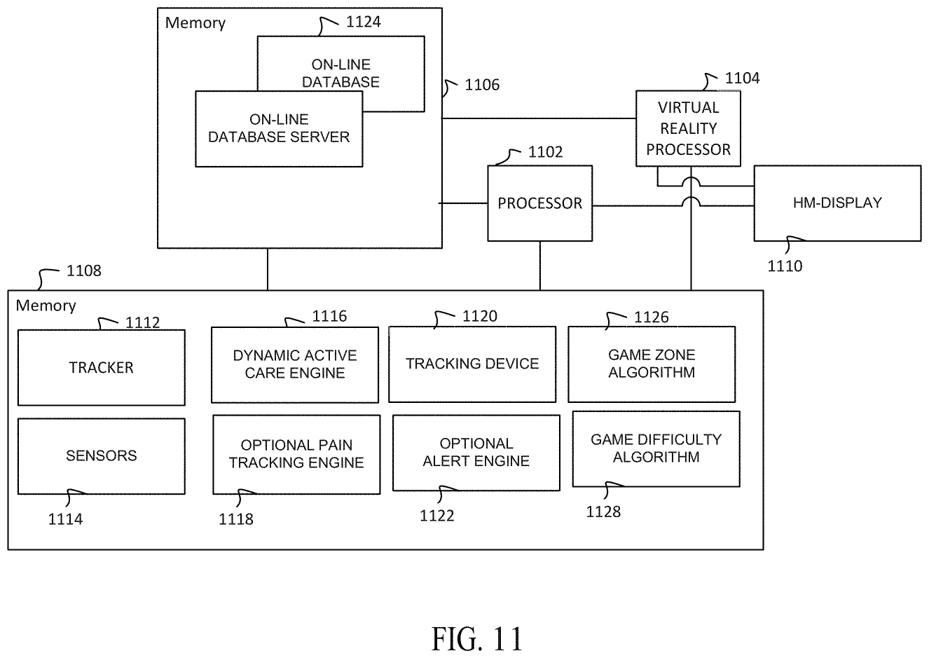

FIG. 11 is a block diagram of a fully automated system that executes the systems (including process flows) and characteristics described herein and those shown in FIGS. 1-10 to provide new and unique treatment plans through virtual rehabilitation. The system comprises processors 1102 and (a virtual reality processor) 1104, a non-transitory computer readable medium such as a memory 1106 and 1108 (the contents of which are accessible to the processors 1102 and 1104), and an Input/output interface (I/O interface—not shown). The I/O interface connects devices and local and/or remote applications such as, for example, additional local and/or remote robotic specifications. The memory 1106 and 1108 stores instructions in a non-transitory media, which when executed by the processor 1102 or 1104, causes the systems and some or all of the functionality associated with the systems to provide virtual rehabilitation, for example. The memory 1106 and 1108 stores software instructions, which when executed by one or both of the processors 1102 and 1104, causes the system to render functionality associated with the virtual worlds and/or virtual environments, a head mounted display 1110, a position tracker 1112, sensors 1114, a dynamic active care plan engine 1116, an optional pain tracking engine 1118, a tracking device 1120 such the optional handheld tracking device, an optional alert engine 1122, an on-line data base 1124, a game play zone algorithm 1126 and a game difficulty level algorithm 1128. In yet another alternate system, the non-transitory media provided functionality is provided through cloud storage. In this system, cloud storage provides ubiquitous access to the system's resources and higher-level services that can be rapidly provisioned over a distributed network. Cloud storage allows for the aggregation of data, which increases the richness of the data used in forming optimal recovery paths and allows for the sharing of resources to achieve consistent services across many monitored devices at many local and remote locations. It further and provides economies of scale.

The memory 1106 and 1108 and/or storage disclosed may retain an ordered listing of executable instructions for implementing the functions described above in a non-transitory computer code. The machine-readable medium may selectively be, but not limited to, an electronic, a magnetic, an optical, an electromagnetic, an infrared, or a semiconductor medium. A non-exhaustive list of examples of a machine-readable medium includes: a portable magnetic or optical disk, a volatile memory, such as a Random-Access Memory (RAM), a Read-Only Memory (ROM), an Erasable Programmable Read-Only Memory (EPROM or Flash memory), or a database management system. The memory 1106 and 1108 may comprise a single device or multiple devices that may be disposed on one or more dedicated memory devices or disposed on a processor or other similar device. An “engine” comprises a hardware processor or a portion of a program executed by a processor that executes or supports unique treatment functions through the disclosed virtual rehabilitation.

When functions, steps, etc. are said to be “responsive to” or occur “in response to” another function or step, etc., the functions or steps necessarily occur as a result of another function or step, etc. It is not sufficient that a function or act merely follow or occur subsequent to another. Further, the systems disclosed herein may be practiced in the absence of any elements not specifically disclosed herein. Some system may be practiced without disclosed elements that are described as alternative elements or alternative systems. The term “substantially” or “about” encompasses a range that is largely (anywhere a range within or a discrete number within a range of ninety-five percent and one-hundred and five percent), but not necessarily wholly, that which is specified. It encompasses all but an insignificant amount.

The disclosed systems rely on computers and wireless sensors to render and display exercises and interfaces to monitor and resolve user actions. In the disclosed systems data flows asynchronously at a sampling rate and frequency that is faster than the physical process it encourages. The high temporal granularity of the volumes of motion track not only rehabilitation progress but also user decisions not to exercise to their capability. The data gathered during the virtual therapeutic sessions can be stored locally or through on-line servers without the user's or therapist's direction. The distributed nature of the systems increase accessibility to user's that cannot travel or do not have access to rehabilitation practices. Further, the distributed nature of the systems allow therapists to track several patients simultaneously from one location and the artificial intelligence may supplement and improve the therapists' judgements increasing the probability of a successful outcome.

Other systems, methods, features and advantages will be, or will become, apparent to one with skill in the art upon examination of the figures and detailed description. It is intended that all such additional systems, methods, features and advantages be included within this description, be within the scope of the disclosure, and be protected by the following claims.

What is claimed is:

1. A non-transitory computer-readable medium having stored thereon a plurality of software instructions that, when executed by a processor, causes:
executing, a physical assessment, by a head-mounted display and a hand-held device, that captures a full volume of motion of an uninjured appendage;
generating, by a machine learning trained engine, a recovery path, care plan, a challenge zone for an injured appendage based on the volume of motion of the uninjured appendage;
initiating a virtual therapeutic session that renders a plurality of simulated virtual environments in the head-mounted display that causes a user to exercise the injured appendage within the challenge zone during the virtual therapeutic session; and
analyzing a user's virtual therapeutic session performance by comparing a user's volume of motion capacity to the full volume of motion.

2. The non-transitory computer-readable medium of claim 1 further comprising detecting a pain level of the user during the virtual therapeutic session and initiating a real-time adjustment to the simulated virtual environments in response.

3. The non-transitory computer-readable medium of claim 1 further comprising mapping in three dimensions the full volume of motion the uninjured appendages during a prior virtual therapeutic session executing the plurality of simulated virtual environments.

4. The non-transitory computer-readable medium of claim 1 where the full volume of motion of an uninjured appendage comprises a target volume of motion.

5. The non-transitory computer-readable medium of claim 1 where the challenge zone comprises a spatial volume in the simulated virtual environment that the user attempts to physically reach.

6. The non-transitory computer-readable medium of claim 1 where movement in the simulated virtual environment comprises a relative starting position.

7. The non-transitory computer-readable medium of claim 1 where the analyzing the user's virtual therapeutic session performance occurs at a sampling rate of about one-hundred frames per second.

8. The non-transitory computer-readable medium of claim 1 where the simulated virtual environments in the head-mounted display that causes the user to move the injured appendage along a predetermined trajectory.

9. The non-transitory computer-readable medium of claim 1 where the challenge zone establishes a plurality of virtual boundaries and a plurality of physical resistance levels.

10. The non-transitory computer-readable medium of claim 1 where the simulated virtual environments prompt the user to execute a reach task.

11. A process comprising:
executing, a physical assessment, by a head-mounted display and a hand-held device, that captures a full volume of motion of an uninjured appendage;
generating a recovery path, care plan, a challenge zone for an injured appendage based on the volume of motion of the uninjured appendage;
initiating a virtual therapeutic session that renders a plurality of simulated virtual environments that causes a user to exercise the injured appendage within the challenge zone during the virtual therapeutic session; and
analyzing a user's virtual therapeutic session performance by comparing a user's volume of motion capacity to the full volume of motion of the uninjured appendage.

12. The process of claim 11 further comprising detecting a pain level of the user during the virtual therapeutic session and initiating a real-time adjustment to the simulated virtual environments in response.

13. The process of claim 11 further comprising mapping in three dimensions the full volume of motion the uninjured appendages during a prior virtual therapeutic session executing the plurality of simulated virtual environments.

14. The process of claim 11 where the full volume of motion of an uninjured appendage comprises a target volume of motion.

15. The process of claim 11 where the challenge zone comprises a spatial volume in the simulated virtual environment that the user attempts to physically reach.

16. The process of claim 11 where movement in the simulated virtual environment comprises a relative starting position.

17. The process of claim 11 where the analyzing of the user's virtual therapeutic session performance occurs at a sampling rate of about one-hundred frames per second.

18. The process of claim 11 where the simulated virtual environments in the head-mounted display that causes the user to move the injured appendage along a predetermined trajectory.

19. The process of claim 11 where the challenge zone establishes a plurality of virtual boundaries and a plurality of physical resistance levels.

20. The process of claim 11 where the simulated virtual environments prompt the user to execute a reach task.
